(19) United States
(12) Patent Application Publication
(10) Pub. No.: US 2007/0123762 A1
(43) Pub. Date: May 31, 2007

(54) Non-invasive spectroscopy of mammalian tissues

(76) Inventors: Gregory P. Crawford, Providence, RI (US); Gregory D. Jay, Norfolk, MA (US)

Correspondence Address: HARRINGTON & SMITH, PC, 4 RESEARCH DRIVE, SHELTON, CT, 06484-6212, US

(21) Appl. No.: 10/587,777

(22) PCT Filed: Jan. 28, 2005
(86) PCT No.: PCT/US05/04834

§ 371 Date: Jul. 28, 2006

Related U.S. Application Data
(60) Provisional application No. 60/539,972, filed on Jan. 30, 2004.

(51) Int. Cl. A61B 5/00; G01N 33/48

(52) U.S. Cl. 600/328; 356/40

(57) ABSTRACT

In the spectrometric device of this invention the objective lens and diffraction grating are replaced with a spectral imaging apparatus on electrically switchable color filter technology. The spectrometric device includes wavelength filter means as the spectral imaging apparatus for transmitting or reflecting wavelengths of light, light intensity sensor means arranged and disposed to measure the intensity of the wavelengths transmitted or reflected by the wavelength filter means and generate an electrical signal from the wavelengths transmitted or reflected, output processing means connected to the light intensity sensor means to receive and process the output from the light intensity sensor means, and display means connected to the output processing means to display the output.

### FIELD OF THE INVENTION

This invention relates to the non-invasive, spectrometric, assessment of hemoglobin in the blood of mammalian tissues.

### BACKGROUND

The assessment of blood in mammalian tissues is important in different scientific disciplines. In medicine, the assessment of blood, and in particular, the assessment of hemoglobin concentration in the blood, is important in the diagnosis and treatment of many diseases and dysfunctions. In forensic science, the assessment of blood is an indication of contusions or bruises of the skin, which are typical consequences of blunt impact trauma. The invention concerns the non-invasive, spectrometric, assessment of hemoglobin concentration in blood in mammals. Two significant applications of this technology are in the diagnosis of anemia, in which hemoglobin concentration is assessed, and in the determination of blunt force trauma, in which hemoglobin degradation and aging is assessed. These two applications are discussed below.

A. Anemia

Anemia is often perceived by the general population to be a minor medical condition. However, according to the World Health Organization (WHO), anemia is the single, largest global illness adversely affecting mortality and worker capacity. The United States Department of Health & Human Services deems it a significant public health concern. Of the 16 million people estimated to have anemia in the United States, 78% go undiagnosed. In developing countries where nutritional inadequacies and infectious diseases are more prevalent, the situation is estimated to be even worse.

Anemia is a deficiency in the number of healthy red blood cells in an individual's body. This deficiency results in oxygen deficiency in the body's tissues and organ systems. Medically, anemia is defined by the WHO as a hemoglobin concentration below 12 g/dL for females and below 13 g/dL for males. Anemia is well known to the general public to influence physical function by causing fatigue and weakness. It also decreases myocardial function and increases peripheral arterial vasodilation and activation of the sympathetic and reninangiotensin-aldosterone system, which strongly influences the initiation or progression of diseases such as heart failure and renal failure. In addition, anemia affects individuals with other diseases; at least 33% of cancer patients, 65-95% of HIV/AIDS patients, and 70% of rheumatoid arthritis patients also have anemia.

Age-related disability and loss in physical function are mounting public health concerns. Loss of physical function endangers the quality of life and independence of many older adults and has significant social and economic repercussions. The prevalence of anemia increases with age and averages about 13% in persons' over 70 years of age. A majority of the anemia in aging adults signifies diseases such as cancer and infectious ailments or are due to iron deficiency or malnutrition. Recent studies indicate that anemia in aging adults ins an independent risk factor for decline in physical performance and is associated with higher mortality risks.

Yet anemia is severely under-diagnosed. The reasons are two-fold. To determine if a patient is anemic, the physician can either make a visual inspection of the palpebral conjunctiva of the eye socket or take a blood sample and have a cell blood count (CBC) test run. Visual inspection, dependent on the physician's experience and training, is at best only 70% accurate and it has been shown that physicians today are less accurate in diagnosing anemia by visual inspection that in the past. Hung, et al., Evaluation of the Physician's Ability to Recognize the Presence or Absence of Anemia, Fever, and Jaundice, Academic Emergency Medicine 7: 746-56 (2000). The CBC test is very accurate, but painful and expensive to perform, time-consuming and often not included in a routine examination.

Because of the need to assess hemoglobin concentration in a more accurate and effective manner, a number of devices and methods have been proposed. Exemplary are (i) retinal imaging, see U.S. Pat. No. 6,305,804 entitled Non-Invasive Measurement of Blood Component Using Retinal Imaging, (ii) blood oxygenation monitoring, see U.S. Pat. No. 6,456,862 entitled Method for Non-Invasive Spectrophotometric Blood Oxygenation Monitoring and U.S. Pat. No. 6,149,589 entitled On-Line and Real-Time Spectoreflectometry Measurement of Oxygenation in Patients Eye, (iii) in-vivo imaging of blood, see U.S. Pat. No. 6,104,939 entitled Method and Apparatus for Reflected Imaging Analysis, (iv) blood analyzer technology, see U.S. Pat. No. 5,791,345 entitled Non-Invasive Blood Analyzer, and (v) image processing of blood vessels, see U.S. Pat. No. 4,998,533 entitled Apparatus and Method for In Vivo Analysis of Red and White Blood Cell Indices. None of these proposed solutions has been embraced by health care professionals, predominately because of inconvenience and inaccuracies in the results.

United States Patent Application Publication No. 2003/0002772, published Jan. 2, 2003 and entitled Non-Invasive Determination of Blood Components discloses a method for estimating an amount of hemoglobin in bodily fluids from the color of a tissue surface of a subject by taking a digital color image of the surface of the subject and a color reference object, decomposing the images into sub-images of component colors having values corresponding to the pixels of the images, selecting a window of each of the images, and calculating an estimated level of hemoglobin using values associated with the component values corresponding to the windows. Although this approach is rapid, due to the inherent noise in this approach, the accuracy of this method is limited.

Accordingly, there is still a significant need in the art for a device that can quickly, accurately and non-invasively measure or assess hemoglobin concentration. Such a device would have many health care applications, such as in routine physical examinations, in emergency rooms, for emergency rescue professionals, during surgery for in-situ measurement of bleeding, for home health care for the chronically ill and aging population, in developing countries lacking medical facilities, in military medical units and in mass casualty situations and triage units, and by oncology, pediatric, obstetric and gynecology, anesthesiology, infectious disease, gastroenterology, cardiology, nephrology, geriatric and urology specialists who deal with anemia on a regular basis.

B. Forensics

Visible contusions or bruises of the skin, the typical consequence of blunt impact trauma, develop after the rupture of blood vessels due to compressive or shearing forces imposed on the body. Bruises are characterized as either subcutaneous or intracutaneous, depending on the tissue layer that is affected. See Bohnert, et al., Spectrophotometric Evaluation of the Colour of Intra- and Subcutaneous Bruises, Int'l Journal of Legal Medicine 113(6): 343-8 (2000). A subcutaneous bruise appears at the site of impact or indirectly by local expansion or shifting of a hemorrhage. After a time interval of hours to days, hemorrhages that are originally localized deep in the tissue layers can extend toward the surface of the skin. The bruise can change color over the course of time from blue to green to yellow during the passing days as a result of the breakdown and diffusion of hemoglobin. When the skin is forced by the application of pressure into channel or profile, the blood is forced into those sections of skin not exposed to the pressure, with hemorrhages occurring as a result of vascular ruptures in the dermis. There have been many studies on how to date bruising and how to distinguish abusive bruising from accidental bruising. See for example, Bariciak, et al., Dating Bruises in Children: an Assessment of Physician Accuracy, Pediatrics 112(4), 804-7 (2003); Dunstan, et al., A Scoring System for Bruise Patterns: A Tool for Identifying Abuse”, Archives of Disease in Childhood 86(5): 330-33 (2002) and Carpenter, The Prevalence and distribution of Bruising in Babies, Archives of Disease in Childhood 80: 363-66 (1999). The color impression of a bruise, its spectral signature and the extent of hemoglobin degradation are all indicators of bruise age. Although a few recent studies of spectrometric assessment of skin have been undertal(en to diagnose various skin diseases, we are aware of no studies relating to spectrometric assessment of skin to assess bruise condition and age. Assessments are routinely made by physicians without evidence-based, scientific, support and there is currently no objective standard or device for assessing bruises in mammals, especially humans.

Accordingly, there is also a compelling need in the art for a non-invasive device and method to accurately age and assess the condition of bruised tissue.

### INVENTION SUMMARY

In one aspect, the invention provides a non-invasive spectrometric device and method for assessing or detecting hemoglobin concentration in mammalian tissues. More specifically, the invention provides a non-invasive spectrometric device and method for assessing or detecting hemoglobin concentration in dermal and epidermal tissues of the skin in mammals. The device can be used to detect hemoglobin concentration in an area of the sl(in that has been subjected to bruising, in the palpebral conjunctiva area of the eye socket, in the earlobe or in any other tissue surface.

Spectrometers are well know in the art. In operation, light energy from a light source enters the spectrometer via an entrance slit, passes through an objective lens, a diffraction grating and an exit slit. The diffraction grating diffracts the light into its component wavelengths and the wavelengths then strikes a detector that generates a voltage in proportion to the intensity of the light hitting it. The voltage drives a read-out device designed to provide the data on the light's intensity. In early spectrometers, only one wavelength could be detected and its intensity measured at a time. More recently, the exit slit and detector have been replaced by an array of charge coupled devices (CCDs), which has enabled measurement of more than one wavelength at a time. (The number of wavelengths that can be simultaneously measured is determined by the number of elements in the CCD array.) The array generates an output that is used to reconstruct the intensity of the light striking each element of the array. This output is sent to a output device such as a monitor, a laptop computer, a PDA (portable digital assistant) device, a printer or the like.

In the spectrometric device of this invention the objective lens and diffraction grating are replaced with a spectral imaging apparatus based on electrically switchable color filter technology. The spectrometric device of the invention comprises wavelength filter means as the spectral imaging apparatus for transmitting or reflecting wavelengths of light, light intensity sensor means arranged and disposed to measure the intensity of the wavelengths transmitted or reflected by the wavelength filter means and generate an electrical signal from the wavelengths transmitted or reflected, output processing means connected to the light intensity sensor means to receive and process the output from the light intensity sensor means, and display means connected to the output processing means to display the output.

In one aspect, the light intensity sensor means is arranged and disposed in stacked relation to the wavelength filter means such that wavelengths of light are transmitted through the wavelength filter means into the light intensity sensor means. In another aspect, the light intensity sensor means is arranged and disposed in angular relation to the wavelength filter means such that wavelengths of light are reflected from the wavelength filter means into the light intensity sensor means. In both of these aspects the light intensity sensor means may be provided by an array of charged coupled devices (CCD) or by a photodiode. The currently preferred embodiment employs a CCD array.

The wavelength filter means comprises at least one pair of planer substrates in parallel-opposed relation, at least one layer of light-wavelength modulating material disposed between the pair of planer substrates to achieve spectral coverage in the visible light spectrum, and a power source in power-providing communication with the substrate. The substrates will typically be composed of ITO-coated glass or plastic such that electricity may be employed as the source of power, but in one aspect of the invention described in detail below, electrically conducting substrates are unnecessary because the source of power is thermal. Three different types of known light-wavelength modulating materials may be employed in the wavelength filter means: deformed helix ferroelectric liquid crystals, holographic polymer dispersed liquid crystals, and cholesteric liquid crystals.

The light-wavelength modulating material, in one aspect, comprises deformed helix ferroelectric liquid crystals (DH-FLC), electrically tuned to exhibit pre-determined wavelength selection properties. By “electrically tuned” we mean that when a voltage is applied across the DH-FLC, the pitch of molecules elongates, which correspondingly lengthens the wavelength of light exhibited. As in understood in the art, the voltage applied to DH-FLC crystals varies the pitch, which lengthens the wavelength of light transmitted or reflected. Due to this fact, varying voltages can be applied to the DH-FLC materials to set the materials to transmit or reflect at pre-determined wavelengths. Typically DH-FLC have been employed in display applications. In such applications, parallel boundary conditions are employed. In the DH-FLC of this inventions, the molecules in the layers of the DH-FLC employed are aligned perpendicular to the surfaces of the planer substrates, i.e. homeotropic alignment. This modification achieves the reflective capacity of the material.

The power source employed to modulate the DH-FLC can be either electrical power or thermal power. For thermal power applications, a transparent resistive heater or other thermal power source is positioned on the planer, exterior, surface of one of the substrates, which are not ITO coated. For electrical power applications, the electrical power source is connected to the conducting elements, the ITO coating, of the substrate to create an in-plane electric field using well-l(now techniques in the art.

In another aspect, the spectrometric device of the invention includes light-wavelength modulating material composed of holographic polymer dispersed liquid crystals (H-PDLC) disposed between electrically conducting substrates. In this aspect, the light wavelength modulating material and electrically conducting substrates are arranged in a stack. The stack is composed of a plurality of layers of H-PDLC arranged in alternating, superposed, relation to a plurality of substrate layers. The number of substrate layers equals the number of layers of H-PDLC, plus one. In other words, the wavelength modulating material includes alternating layers of, from bottom to top, substrate and H-PDLC in a stack with the top layer being a layer of the substrate. Each side of the substrate layer adjacent to H-PDLC will have an electrical conducting coating, for example indium-tin-oxide (ITO) in order to complete the circuit. Consequently, the top and bottom layers of substrate may have an electrical conducting coating on only the side, the side disposed interiorly and adjacent to the H-PDCL. The stack may be composed of as many alternating layers of electrically conducting substrate and H-PDLC as is desired but preferably the stack will be composed of between two and ten layers of H-PDLC (and therefore between three and eleven layers of substrate).

In one embodiment of this aspect of the invention, the stack is composed of one layer of H-PDLC sandwiched between two layers of electrically conducting substrate. In this aspect, there exists in the H-PDCL film a variable index of refraction of the liquid crystal, which is different from the index of refraction of the polymer. This variable index of refraction permits continuous modification of the reflection or transmission peak, thereby eliminating the need for multiple “gratings”, each providing reflection or transmission at a single peak. These variable refraction index H-PDLC and their operation are described in detail in United States Patent Publication No. 2002/0130988 herein incorporated by reference.

In an alternate embodiment of the invention, the light-wavelength modulating material is composed of cholesteric liquid crystals (CLC) disposed between electrically conducting substrates. In this embodiment the CLC may also be composed a plurality of CLC layers arranged in alternating, superposed, relation to a plurality of substrate layer. In this case, the stack will have a number of substrate layers one greater than the number of CLC layers and the power source will produce electrical energy perpendicular to the pitch axis of the CLC layers.

The CLC layers have the capacity to reflect light of different, per-determined wavelengths, but because they are inherently reflect light in a right or left handed manner, the maximum efficiency will be only 50%. Consequently, to increase the efficiency, the device may further include a passive optical element such as a quarter-wave plate disposed in parallel relation between two reflective CLC of opposite-handedness.

In another aspect, one CLC layer may be interposed between two electrically conducting substrate layers. In this aspect, there exists in the CLC film a variable index of refraction of the liquid crystal, which is different from the index of refraction of the polymer. Lil(e the variable refraction index H-PDLC, the variable index of refraction in the CLC permits continuous modification of the reflection or transmission peal(, thereby eliminating the need for multiple “gratings”, each providing reflection or transmission at a single peak.

The output processing means connected to the light intensity sensor means to receive and process the output from the light intensity sensor means can be any of the well-known output processing means employed in spectrometers. Likewise, the display means connected to the output processing means to display the output can be configured using well-known techniques in the art to display indicia of the estimated level of hemoglobin detected.

In the operation of the device of the invention, light is projected from the area of epidermal tissue of interest into the device and is then filtered by the wavelength filter means and detected by the light sensor means of the device, the latter of which generates an electrical signal from the wavelengths transmitted or reflected and transmits that signal to the output processing means, which processes the output and transmits it to a display readable by a physician or other health care professional. The basics of how spectrometers and other spectroscopic tools (such as, for example, spectrophotometers) work is well known in the art and succinctly described in Steven L. Brown, “Laboratory Techniques for General Chemistry, Ch. 5, Spectroscopy”, 2002, Hayden-McNeil Publishing, Plymouth, Mich.

The invention also includes a method of detecting or assessing the concentration of hemoglobin in a mammalian subject suspected of having an abnormal hemoglobin concentration. The method comprises the steps of (a) exposing an area of tissue of a mammalian subject suspected of having an abnormal hemoglobin concentration to a spectrometer of the invention to receive and analyze light reflected from the area of tissue; (b) reading the output from the spectrometer indicating the hemoglobin concentration in the area of tissue exposed to the spectrometer; and (c) comparing the hemoglobin concentration of the output to the hemoglobin concentration in a control standard for a normal epidermal tissue specimen.

### DESCRIPTION OF THE DRAWINGS

FIG. 1 is an illustration of a H-DPLC containing device. In (a) the prior art panel is illustrated in its voltage-off and voltage applied positions. In (b) and (c) two embodiments of the device of the invention are illustrated in which only one layer of H-DPLC is employed.

FIG. 2 is a graphic representation of the transmission results (a) and the reflection results (b) for the stack of five H-DPLC layers described in detail below.

FIG. 3 illustrates the mode of operation of a prior art CLC panel (a) in planar (left), focal conic (middle) and homeotropic (right) states. In (b) an embodiment of the invention composed of a stack of three CLC panels is illustrated. In (c) an embodiment of the invention composed of one CLC in IPS mode is illustrated.

FIG. 4 is a graphic representation of the transmission results (a) and the reflection results (b) for a CLC device composed of three panel pairs to reflect read, green and blue as described in detail below. Results are shown in (c) for a single panel CLC IPS device that nearly covers the entire spectral range as described in detail below.

FIG. 5 illustrates the device of the invention comprising DH-FLC crystals subject to in-plane switching to produce a red-shift.

FIG. 6 is a graphic representation of the transmission results (a) and reflection results (b) as temperature varies in the DH-FLC containing device of the invention.

### DETAILED DESCRIPTION

In the spectrometric device of this invention the objective lens and diffraction grating are replaced with a spectral imaging apparatus based on electrically switchable color filter technology. The spectrometric device of the invention comprises wavelength filter means as the spectral imaging apparatus for transmitting or reflecting wavelengths of light, light intensity sensor means arranged and disposed to measure the intensity of the wavelengths transmitted or reflected by the wavelength filter means and generate an electrical signal from the wavelengths transmitted or reflected, output processing means connected to the light intensity sensor means to receive and process the output from the light intensity sensor means, and display means connected to the output processing means to display the output. The light intensity sensor means may take the form of an array of charge coupled devices (CCDs) or a photodiode. The output processing means and display means are both well-recognized elements of spectrometric devices and need not be described in detail here as the skilled artisan would be able without undue experimentation to arrange, connect and incorporated these elements. Any of the well-known output processing means and display means known in the art may be used.

In one aspect, the light intensity sensor means is arranged and disposed in stacked relation to the wavelength filter means such that wavelengths of light are transmitted through the wavelength filter means into the light intensity sensor means. In another aspect, the light intensity sensor means is arranged and disposed in angular relation to the wavelength filter means such that wavelengths of light are reflected from the wavelength filter means into the light intensity sensor means.

The wavelength filter means comprises at least one pair of planer substrates in parallel-opposed relation, at least one layer of light-wavelength modulating material disposed between the pair of planer substrates to achieve spectral coverage in the visible light spectrum, and a power source in power-providing communication with the substrate. The substrates will typically be composed of ITO-coated glass or plastic such that electricity may be employed as the source of power, but in one aspect of the invention described in detail below, electrically conducting substrates are unnecessary because the source of power is thermal. Three different types of known light-wavelength modulating materials may be employed in the wavelength filter means: holographic polymer dispersed liquid crystals (H-PDLC), cholesteric liquid crystals (CLC), and deformed helix ferroelectric liquid crystals (DH-FLC). These types of liquid crystals are well-known in the art. Their use, including the modifications necessary or desirable to employ them in the spectrometric device of the invention, is described in detail below.

A. H-PDLC Containing Devices

Holographic polymer dispersed liquid crystals are created by a simple one-step fabrication process in which a homogeneous mixture of photosensitive propolymer and nematic liquid crystal is exposed to an interference pattern process following the method disclosed in Bowley and Crawford, “Diffusion Kinetics of Formation of Holographic Polymer Dispersed Liquid Crystal Display Materials”, 2000, Applied Physics Letters 76. In the bright regions of the interference pattern the polymerization occurs more rapidly that in the dark regions, forcing the non-reactive liquid crystal out of the bright regions and into the dark regions. This diffusion process creates a stratified material composed of liquid crystal droplets and polymer rich layers that is locked-in by the photo-polymerization process. The grating pitch is given by Λ=λ_{f/}/2<n>sin θ, where λ_{f/} is the wavelength of the exposing laser beams, <n> is the average index of refraction of the liquid crystal and polymer mixture , and 2 θ is the angle between the exposure beams inside the sample. Since the liquid crystal typically has an average index of refraction that is larger than that of the polymer, a spatial perturbation in the index of refraction exists.

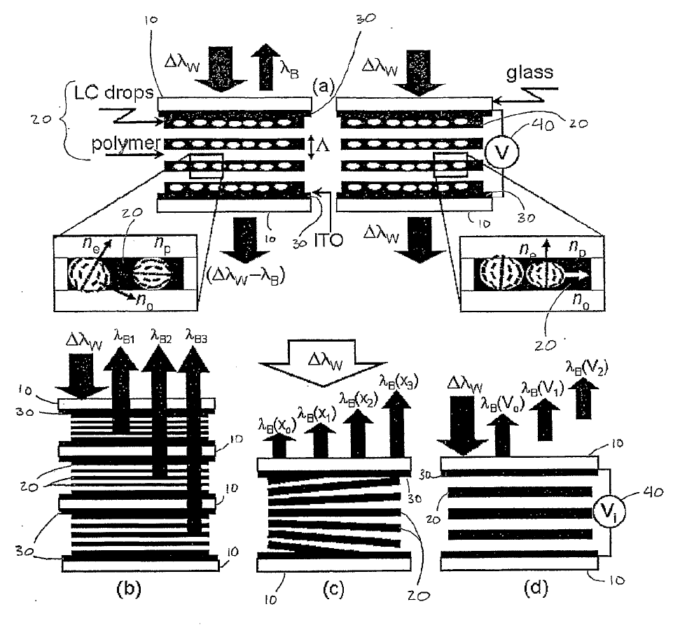

The principle is illustrated in FIG. 1(a). The H-PDLC includes liquid crystal and matrix polymer layers (20) which form a reflection grating capable of reflecting a wavelength of light disposed between a pair or more than one pair of electrically conducting substrates (10), which may be formed from indium-tin-oxide (ITO)-coated glass or plastic. See for example, PCT Patent Publication WO 01/20406 published 22 Mar. 2001, which discloses a multicolored reflection liquid crystal display device in which the liquid crystal film is capable of reflecting two different wavelengths of light. Each electrically conducting substrate layer (10) is connected with a means for providing electrical energy through the electrodes of the conductant (30) and into the H-PDLC (20).

In the absence of an applied voltage, a refractive index modulation exists between the liquid crystal rich planes (shown as droplets) and the pure polymer planes of the H-PDLCs. The average index of refraction of the liquid crystal rich layers, n_{LC}, is some combination of the ordinary, nₒ, and the extraordinary, nₑ, index of refraction of the liquid crystal, which is estimated as n_{LC}²≈(nₑ²+2nₒ²)/3. When the material is illuminated with a broadband white-light source, a narrow reflection band is rejected with reflectives greater than 50% and peak widths in the 15-30 nm range depending on the birefringence of the liquid crystal, index of refraction of the polymer, and sample thickness. Since liquid crystal molecules possess a positive dielectric anisotropy they align parallel to the applied electric field when an external voltage (40) is applied. In the aligned state, the ordinary refractive index of the liquid crystal matches that of the polymer and the index modulation vanishes. The H-PDLC (20) becomes transparent to all wavelengths, as shown in FIG. 1(a), right panel. Switching voltages in the range of 50-100 volts are needed, since the liquid crystal is highly constricted by the holographic planes of dimensions 170-200 nm for visible reflections; however, the response times can be very fast, in some cases, less than 100μ seconds.

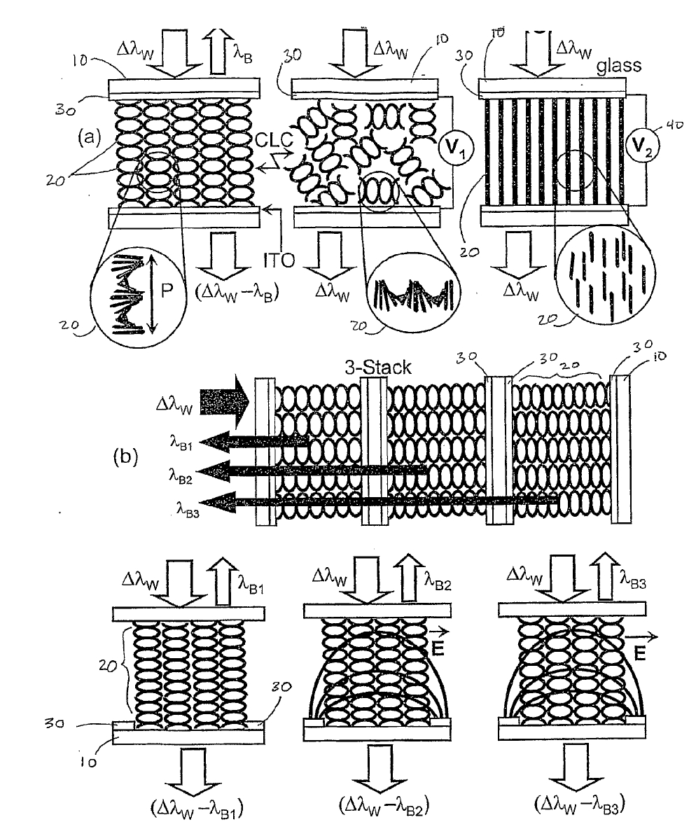

In one embodiment of the current invention, these HPDLCs are employed as a spectrometric device. This embodiment is illustrated in FIG. 3(b), in which the spectrometric device comprises a plurality of planer H-PDLC film layers disposed in alternating, stacking relation with a plurality of planer ITO-coated substrate layers. The H-PDLC stratified films are sandwiched between the ITO coated glass substrates and maintained at a distance of between about 2 to about 30 micronmeters. Between 2 and 20 H-PDLC film layers, and therefore between 3 and 21 substrate layers may be employed. Preferably, between 2 and 10 layers of H-PDLC and therefore between 3 and 11 substrate layers may be employed. Electrical leads 30 are then connected to the edges of each of the planer ITO glass substrates so the H-PDLC material can be exposed to an applied voltage. When a voltage is applied, an electric field is created within the material and the H-PDLCs can be tuned to a transparent state. By using multiple H-PDLC layers, various wavelengths can be allowed to pass through the stack by applying different voltages to the different substrate layers. In FIG. 1(b), the stack is shown with broad band incident white light, Δλ_{W}, and three reflection bands λ_{B1}, λ_{B2}, λ_{B3}, whose peak wavelength is dictated by Bragg's law, λ_{B}=2d<n> for normally incident light, where d is the thicl(ness of the holographid plane. In the embodiment illustrated in FIG. 3(b), a stack of three H-PDLC layers is shown but the device may be constructed with more or less than three layers to generate a number of reflection bands corresponding to the number of H-PDLC layers in the stack.

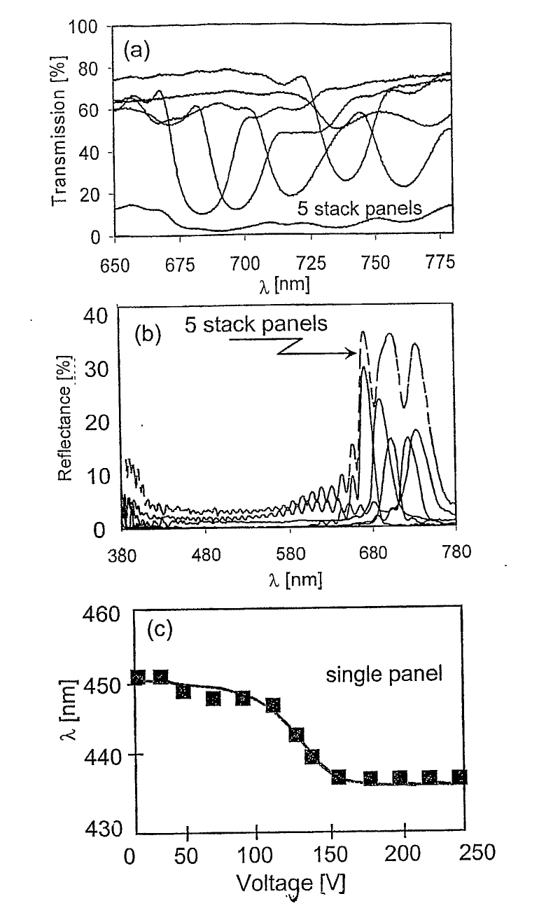

In another embodiment, the stack is composed of five H-PDLC film layers interspersed between planer ITO-coated substrates instead of three layers as described above. FIG. 2 is a graphic representation of data for such a stack in transmission, FIG. 2(a) and in reflection, FIG. 2(b). In reflection (FIG. 2(b)), the five-layered H-PDLC stack exhibits between 30-40% reflectance in the wavelength range between about 600 and 760 λ[nm].

Referring again to FIG. 1, parts (c) and (d) illustrate two alternate embodiments of this aspect of the inventions. In the aspect illustrated in (c), only one H-PDLC film is needed and employed because a spatial gradient has been created in the holographic plane sizes from one edge of the sample to the other. The H-PDLC sample can then act as a wavelength modulating material for multiple wavelengths. In order to selected different wavelengths, the ITO coating can be pixilated so that the various wavelengths can be electrically addressed independently.

In the alternate embodiment shown in (d), only one H-PDLC film is needed and employed because there exists in the H-PDLC film a variable index of refraction of the liquid crystal, which is different from the polymer. In this way, as the electric field is applied to the two substrates, and the index of refraction changes with respect to the index of the polymer enabling the H-PDLC to be electrically addressed to a pre-determined wavelength. This alternate embodiment is described in detail in PCT Patent Publication WO 01/20406 published 22 Mar. 2001, which is herein incorporated by reference.

All of these above embodiments of H-PDLC can be used in the non-invasive, spectrometric device of the invention to assess hemoglobin concentration in the blood of mammalian tissues.

B. CLC Containing Devices

Cholesteric liquid crystals (CLC) are also known in the art. CLC exhibit long-range, orientational order analogous to conventional nematic liquid crystals, except that the molecules are chiral. See Blinoff and Chigrinov, Electro-Optic Effects in Liquid Crystals, Springer, N.Y., (1994). Consequently, the structure acquires a spontaneous right-handed or left-handed twist about a helical axis normal to the average direction of the liquid crystal molecules. The degree of twist of the phase is characterized by the cholesteric pitch, “P”. Heretofore, CLC have been used in flat-panel display applications. See Yang, et al., in Liquid Crystals in Complex Geometries Formed by Polymer and Porous Networl(s, Crawford, GP and Sumer, S, eds., Taylor & Francis, London (1996); Doane, et al., Cholesteric Liquid Crystals for Flexible Display Applications, John Wiley & Sons, London, (2004). The operation of a CLC device is illustrated in FIG. 3(a) left panel. In the zero voltage state illustrated in the left panel, the molecules are aligned in the planar configuration between the substrate layers, and since the structure is periodic, they can reflect a bandwidth centered at λ_{B}, which is dictated by Bragg's law (λ_{B} =<n>P for normal incidence). Upon application of an applied voltage (V₁˜10-15 volts for a 5 μm sample), a positive dielectric anisotropy material (Δε>0) transforms to a focal conic state, which is characterized by a random distribution of helical pitches, as shown in FIG. 3(a), middle panel. This state is transparent and remains that way even after the voltage is removed. In other words, this device possesses bi-stable memory since the focal conic state can remain indefinitely even after the field is removed. Upon application of a higher voltage (V₂˜25-30 volts), the material transforms to the hometropic, aligned, state as shown in FIG. 3(a), right panel. When the voltage is abruptly removed, the homeotropic state transforms back to the reflective planar state. FIG. 3(a) right panel. The chiral pitch can be engineered, or set, by mixing in different concentrations the chiral components to reflect in the ultraviolet, visible and near infrared in accordance with art-recognized techniques. The switching time is on the order of 30-50 ms slower, depending on the mode, than other liquid crystal materials.

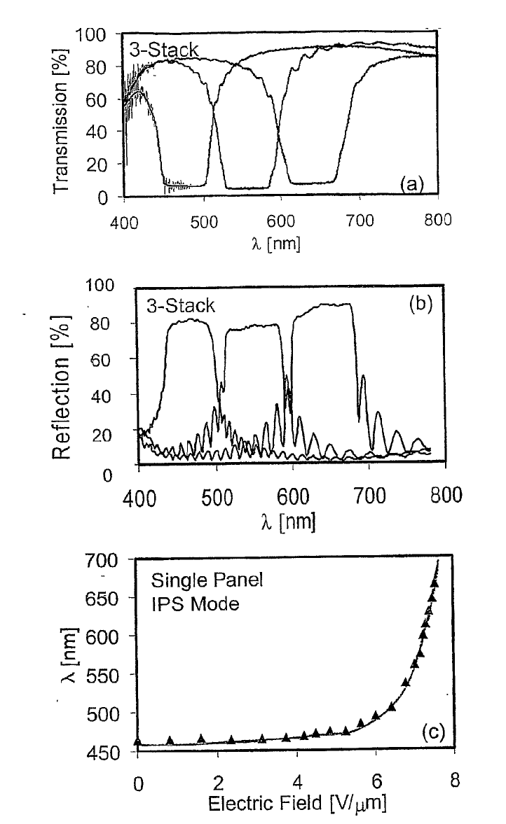

In order for CLC to be used in a non-invasive spectrometer, full spectral coverage in the visible light spectrum is required. This can be achieved by employing a CLC stack, comprising a plurality of layers of cholesteric liquid crystal materials. FIG. 3(b) illustrates a three-stack of CLC panels that reflect red, green and blue. FIG. 4(a) presents the data for this stack in the transmission mode and FIG. 4(b) presents the data for this stack in the reflection mode upon application of the applied voltage. Since CLC are intrinsically right- or left-handed because of their chirality, they can reflect only right-handed or left-handed, circularly polarized, light, ideally with 50% efficiency at the Bragg wavelength. To solve this problem, we disposed and arranged the panels that reflected red, green and blue as panel pairs, each color having a right handed and a left handed panel forming the respective pairs, so that we actually had six panels in the stack, two red, then two green, then two blue. We then integrated a passive optical element between two identically reflecting panels to achieve a much higher efficiency, exceeding 80%, as shown in FIG. 4(a) and (b). In the embodiment illustrated the passive optical element employed was a quarter-wave plate, but the skilled artisan will recognize that any passive optical element could be used. Alternatively, to achieve the same outcome, a left handed panel and a right handed panel with the same λ_{B} can be stacked.

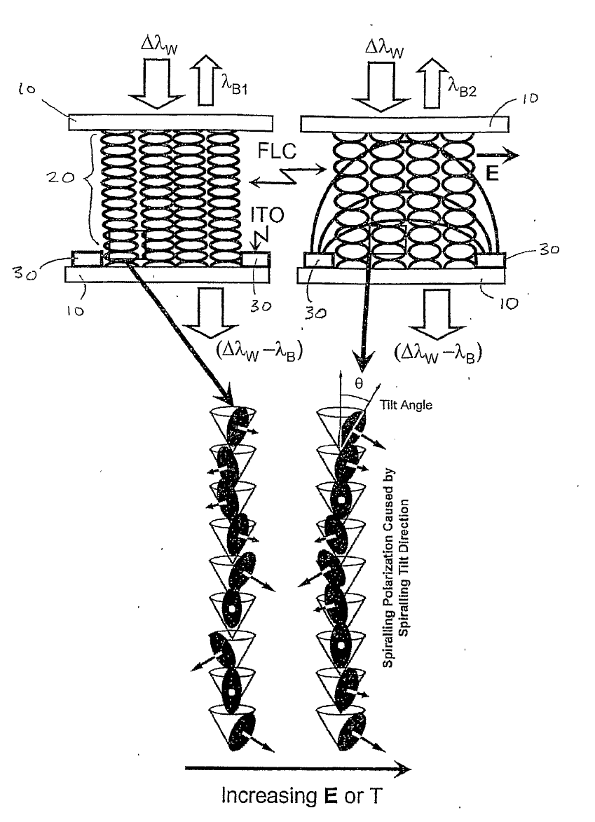

The stacked solution illustrated in FIG. 4(b) was experimentally evaluated and the results are shown in FIG. 5(a) and (b). The full spectral width of the reflection peak in FIG. 4(b) is Δλ_{FWHM}>100 nm. The spectral width is largely dictated by the birefringence of the CLC material, Δn. To decrease the spectral bandwidth, materials with a lower An can be employed. For example, for a material with a Δn˜0.05, bandwidths on the order of Δλ_{FWHM}˜30 nm for λ_{B}=600 nm can be achieved. Because CLC materials do not have the scattering losses observed with H-PDLC materials, numerous panels can be stacked to achieve full spectral coverage in the visible range.

A CLC in-plane switching (IPS) mode is shown in FIG. 4(c) in which field direction is parallel to the substrates, orthogonal to how it was applied in FIG. 4(a) and (b). As an in-plane voltage is applied perpendicular to the pitch axis of the CLC, the pitch elongates and the reflection red-shifts. This IPS mode permits the use of one panel to span nearly the entire visible spectral range, as is shown in FIG. 4(c). This mode enable electrical tuning of the reflection band from 450 nm to 700 nm. Assuming linearity in the transition region, the tunability/resolution metric is ΔV/Δλ˜0.15 V/nm for the peak maximum. The downside of this approach is that overall efficiency may be reduced because the electrodes on the surface of the substrate reduce the optical throughput, as CLC materials do not respond above the electrode. By offsetting a pair of transparent conducting electrodes on the top and bottom substrates and by driving them with voltage signals out of phase, this problem is alleviated. In addition, because the switching voltage is higher in the CLC IPS mode as compared to conventional IPS mode, the electrodes must be arranged closer together so that maximum drive voltages do not exceed 40-50 volts. The preferred distance between electrodes is around 5 μm, but the skilled artisan will recognize that distance between electrodes can be optimized for various materials without any undue experimentation.

C. DH-FLC

The art-known ferroelectric liquid crystal, or chiral smectic C phase (SmC) liquid crystals, consists of layers of molecules. This thickness of the chiral smectic C layers are less than one molecular length. See Yeh and Gu, Optics of Liquid Crystals, John Wiley, New York, (1999). As a result, the molecules must tilt at an angle with respect to the layer normal. Because the tilt angle is fixed, the molecular orientation is confined to a cone with a half apex angle of θ. Ferroelectric liquid crystal materials have intrinsic chirality and associated pitch, much lile CLC materials, and they have a dipole moment perpendicular to the long molecular axis, rather than parallel to it as in the case of CLCs materials. In ferroelectric switching, the molecules switch on the cone.

Art-known, deformed helix ferroelectric liquid crystal materials (DH-FLC) were first used in display applications. See Verhulst, et al., A Wide Viewing Angle Video Display Based on Deformed Helix Ferroelectric LC and Diode Active Matrix, Proceeding of the International Display Research Conference 94: 377-80 (1994). In those applications, parallel boundary conditions were employed, i.e., the molecules are aligned in parallel to the substrate surfaces In this embodiment of the invention, homeotropic alignment, wherein the molecules are aligned perpendicular to the substrate surfaces, should be employed so that the panel provides a wavelength selection property similar to the CLC panel. Dynamic switching times for panels of DH-FLC materials operated as described above should be less than 500 μs.

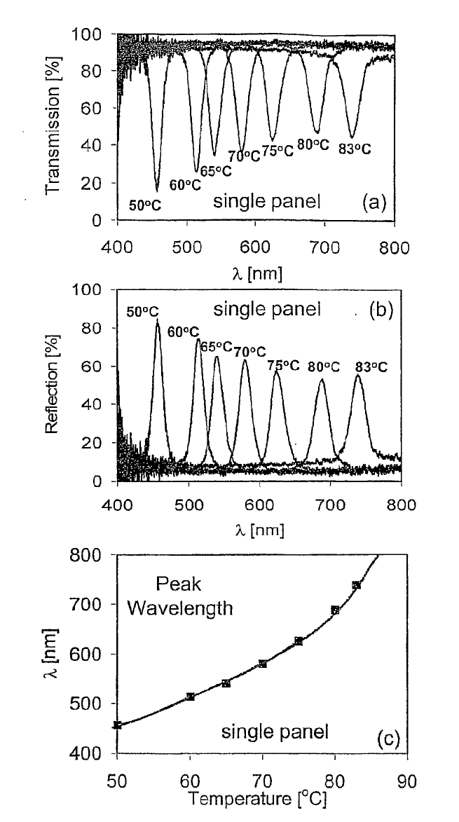

This embodiment is illustrated in FIG. 5. Pitch is deformable using temperature or an in-plane electric field. If an in-plane electric field is employed, the helix deforms or elongates and the reflection red-shifts. If the in-plane switching (IPS) mode described above for CLC materials is employed, the same disadvantages described will result and accordingly, the IPS electrodes should be located closer together to minimize aperture loss. A preferred range is about 5 μm, but the skilled artisan will recognize that distance between electrodes can be optimized for various materials without any undue experimentation. If temperature is employed to accomplish switching, a transparent resistive heater may be incorporated into the device and employed in place of electric power in accordance with well-recognized methods. A DH-FLC panel having homeotropic alignment at the surfaces, i.e., in which the molecules are aligned perpendicular to the plane of the layer was prepared and subjected to temperature increases. The data is presented in FIG. 6(a) and (b) for transmission and reflection, respectively. Since the refractive index of the material was small and the cone angle was around 30 degrees, the sample was tilted with respect to the incident light to increase the index perturbation with respect to the incoming light. One DH-FLC panel can cover the entire visible spectral range, as is illustrated in FIG. 6(c). Assuming linearity in the transition region if FIG. 6(c), the tenability/resolution metric is ΔV/Δλ˜0.12°/nm.

The DH-FLC panels of the invention may be switched either electrically or thermally. Electrical switching is currently the preferred mode. Like the CLC-containing devices of the invention , electrical switching may be accomplished by advantageous employment of the herein disclosed IPS mode, in which the field direction is parallel to the substrates. Thermal switching can be accomplished by employing a transparent resistive heater, which is connected to a power supply (typically a current source) employing methods and materials well-know in the art.

Other embodiments are within the scope and spirit of the claims. Certain elements and functions of the invention described above can be implemented using software, hardware, firmware, hardwiring, or any combinations of these in art-recognized ways. Features, elements and means of the invention implementing various functions may be physically located at various positions rather than in a single location or apparatus.

All references cited in this document are hereby incorporated by reference herein for the substance of their disclosure.

We claim:

1. A non-invasive spectrometric device for assessing the level of hemoglobin in mammalian tissues comprising (a) wavelength filter means for transmitting or reflecting wavelengths of light; (b) light intensity sensor means arranged and disposed to measure the intensity of the wavelengths transmitted or reflected by the wavelength filter means and generate an electrical signal therefrom, (c) output processing means connected to the light intensity sensor means to receive and process the output therefrom; and (d) display means connected to the output processing means to display the output.

2. The device of claim 1 wherein the light intensity sensor means is arranged and disposed in stacked relation to the wavelength filter means such that wavelengths of light are transmitted through the wavelength filter means into the light intensity sensor means.

3. The device of claim 1 wherein the light intensity sensor means is arranged and disposed in angular relation to the wavelength filter means such that wavelengths of light are reflected from the wavelength filter means into the light intensity sensor means.

4. The device of claim 1 wherein the wavelength filter means comprises at least one pair of planer substrates in parallel-opposed relation, at least one layer of light-wavelength modulating material disposed between the pair of planer substrates to achieve spectral coverage in the visible light spectrum, and a power source in power-providing communication with the substrate.

5. The device of claim 4 wherein the substrates are electrically conducting substrates.

6. The device of claim 4 wherein the light-wavelength modulating material comprises deformed helix ferroelectric liquid crystals (DH-FLC), electrically tuned to exhibit pre-determined wavelength selection properties.

7. The device of claim 6 wherein the molecules in the layers of the DH-FLC are aligned perpendicular to the surfaces of the planer substrates.

8. The device of claim 5 wherein the power source is in electrical communication with the substrates to create an in-plane electric field.

9. The device of claim 4 wherein the power source is in thermal communication with one of the pair of substrates to create a temperature change in the wavelength modulating material.

10. The device of claim 9 wherein the power source is a transparent resistive heater positioned on the planer exterior surface of one of the pair of substrates.

11. The device of claim 5 wherein the light-wavelength modulating material comprises a layer of holographic polymer dispersed liquid crystals (H-PDLC).

12. The device of claim 11 wherein one layer of H-PDLC is arranged between two parallel-opposed electrically conducting substrate layers so as to form a spatial gradient in the H-PDLC from one edge of the substrate layers to the opposing edge of the substrate layers.

13. The device of claim 11 wherein one layer of H-PDLC is arranged between two parallel-opposed electrically conducting substrate layers and wherein the H-PDLC has an index of refraction variable in response to an applied electric field.

14. The device of claim 11 comprising a stack composed of a plurality of layers of H-PDLC arranged in alternating, superposed, relation to a plurality of substrate layers, wherein the number of substrate layers equals the number of layers of H-PDLC plus one.

15. The device of claim 12 wherein the stack is composed of between two and twenty layers of H-PDLC layers.

16. The device of claim 5 wherein the light-wavelength modulating material comprises at least on layer of cholesteric liquid crystals (CLC).

17. The device of claim 14 forming a stack composed a plurality of CLC layers arranged in alternating, superposed, relation to a plurality of substrate layers, the plurality of CLC layers having the capacity to reflect light of different, per-determined wavelengths, the stack having a number of substrate layers one greater than the number of CLC layers and wherein the power source produces electrical energy perpendicular to the pitch axis of the CLC layers.

18. The device of claim 15 further comprising a passive optical element disposed in parallel relation between two reflective CLC of opposite-handedness.

19. The device of claim 16, composed of one layer of CLC disposed between two layers of electrically conducting substrate, wherein the one layer of CLC is subjected to a in-plane electric field to produce different pitch sizes as the electric field is increased.

20. The device of claim wherein the light intensity sensor means is selected from the group consisting of an array of CCD and a photodiode.
